(19) United States
(12) Patent Application Publication
(10) Pub. No.: US 2003/0120174 A1
(43) Pub. Date: Jun. 26, 2003

(54) Thermostimulating device for thermographic investigations in dermatology, oncology, angiology and capillaroscopy

(76) Inventors: Ferdinando Ippolito, Rome (IT); Aldo Di Carlo, Rome (IT)

Correspondence Address: YOUNG & THOMPSON, 745 SOUTH 23RD STREET 2ND FLOOR, ARLINGTON, VA, 22202

(21) Appl. No.: 10/322,540

(22) Filed: Dec. 19, 2002

(30) Foreign Application Priority Data
Dec. 21, 2001 (IT) RM2001A000764

(51) Int. Cl. A61B 5/00

(52) U.S. Cl. 600/555

(57) ABSTRACT

A thermostimulation device is disclosed, said device being intended for application to thermographic investigation and allowing sensitivity and specificity in the clinical field of such investigation to be improved, in particular for dermatology, oncology, angiology and capillaroscopy. The finding comprises a probe whose inside is discontinuously supplied with a refrigerating or a heating liquid mixture that comes from a reservoir where it is kept at the temperature that has been set forth previously for the specific test, respectively +5° C. in case of cryostimulation and +40° C. in case of “hot” stimulation. The introduction of the mixture into the probe is adjusted by means of a valve whose opening time is controlled by the operator, so as to determine the passage of a predetermined volume of the mixture immediately before proceeding to thermal stimulation. At the end of thermal stimulation the liquid is sent back from the probe to the reservoir.

It is the object of this invention a thermostimulating device that can be employed for thermographic investigation in dermatology, oncology, angiology and capillaroscopy and more particularly the object of this invention consists in a thermogenic probe, or a small rubber balloon containing a 50% water-alcohol mixture that is put in contact with the skin area to be examined for 20″. The temperature of the mixture is previously set at the value of +5° C. (cryostimulation) or of +40° C. (hot stimulation). This is done with the aim of evaluating more exactly the thermographic gradients by means of the detection of the respective times of thermal recovery after thermal stimulation, instead of employing the direct thermographic observation.

The telethermographic investigation employed in the study of cutaneous tumors has resulted in data of undoubtful interest both as far as the clinical standpoint is concerned, showing to be a useful complement for the diagnostic procedure and for checking in the lapse of time any suspectable lesions, and as to the experimental standpoint: for instance, in the case of melanoma, the finding of a tumoral hyperthermic image, the perilesion hyperthermic halo, the lymphocentric hot striae are important data which deserve futher deepening. Data obtained in the latest years through numerous observations carried out by means of telethermography on patients affected by cutaneous neoplastic manifestations have confirmed the usefulness of such methodology. Examinations of the superficial circulation, both of large and of small blood vessels, have confirmed the usefulness of such methodology also in the field of angiopathies and in capillaroscopy. Indeed, skin as a coating organ, allows a more authoritative exploration with respect to other deep organs.

On the other side, it has been observed since a long time that the employment, during telethermographic investigations, of the cooling or heating of tissues to be examined using the parameters mentioned above (thermostimulation: +5° C. or +40° C.) the thermal gradients in the area are temporarily modified without any biological damage to tissues, and that a very sharp visualization of the specific thermographic frame is obtained. This occurred as a result of the objective differences in the thermal recovery time (TRT) even in the case of basic thermal gradients differences of very low value, and even lower than 0.1° C., the technical limit of thermographic devices of the present time. TRTs are definitely the expression of thermogenic potentials of tumoral lesions or of the conditions of cutaneous irroration. For instance, in the case of ischemic angiopathies, the employment of a hot stimulus applied to the cutaneous region under examination allows the evaluation to be carried out of ischemia or the hypoflow into the thermostimulated cutaneous area. Thermostimulation has been employed since the time of its first applications in the telethermographic study but in a very empirical way, for instance employing a hair dryer or by dipping the part to be tested into cold/warm water and it has been little employed because it was badly affected both by the non-standardized distance from the position where the hair dryer was held, and by forced evaporation, as well as by the residual presence of water on the cutaneous surface etc. Moreover, said procedures do not allow some physical variables to be controlled with certainty, such as the degree of thermal stimulus in ° C., the duration of said thermal stimulus, the thermal recovery times.

In the Italian patent no. 1.171.306, a cryostimulation device has been made known comprising a probe containing a refrigerating liquid mixture, whose cooling occurs through the Joule-Thomson effect by means of the expansion of a compressed gas inside the probe itself. The temperature of the refrigerating mixture within the probe is measured by a thermocouple and an in-flow valve which allows more gas to be automatically introduced that, by expansion inside the probe itself, cools the walls of the chamber containing the mixture, keeping it at the desired temperature. Such a device however shows some meaningful limitations: first of all, it can be employed just for the cold stimulation and not for the hot stimulation. Moreover, the mixture contained within the probe is kept at a constant temperature so that the thermal exchange between the patient's skin and the probe mixture becomes modified during the thermal stimulation (t₀−t₁): thus the mixture temperature is the same, both at the time t₀ (the time of starting the thermal stimulation) and at the time t₁ (the end of thermal stimulation): such data do not allow the test to be repeated.

Another device that has already been known in the literature regards the employment of a probe in which a water-alcohol refrigerating solution is circulated continuously. Such solution comes to the probe through a two-way pipe from a reservoir where it is kept at a constant temperature by means of a suitable heating/cooling internal system and is pushed at a high speed into the probe by means of a pump. In this case also the solution is not satisfying as it involves a continuous thermal exchange among the inlet liquid of the probe, the outlet liquid of the probe and the patient's skin, with the result of the same drawbacks as those previously stressed.

It is the task of the present finding the removal of such drawbacks through the proposal of the realization of a simple and cheap thermostimulation apparatus, by means of which the heat amount that is exchanged with the patient's skin is predetermined. This has been obtained according to the present invention through the employment of a thermogenerating apparatus endowed with a special probe. The 50% water-alcohol mixture previously cooled down to +5° C. or heated up to +40° C. is conveyed to the head of the thermal probe, before stimulation, for a predetermined time and then it is applied without any pressure to the skin area for 20″.

In that way, this invention allows a constant starting temperature at t₀, by means of a device that stops the liquid inlet at the time t₁, to be obtained, in addition to the fixed parameters already obtainable for a correct stimulation. Such data allows the examination to be repeated under any examination conditions.

In particular, according to the present finding, said probe is made up of a thin, flexible rubber membrane, in which a heating/cooling liquid mixture is introduced, said mixture being made up of, for instance, water and alcohol in equal parts. The mixture is contained within a reservoir where it is kept at a temperature which is stabilized by heating/cooling means that are already known per se. The mixture is conveyed, by means of a pump placed inside the probe, through a valve whose opening is controlled by the operator, for a predetermined time so as to determine the passage of a predetermined volume of the mixture, immediately before carrying out the thermal stimulation. When the application is over, the operator can empty the probe by his/her command, so causing the filling mxture to flow back to the reservoir.

The temperature of the mixture itself within the reservoir is controlled through a thermocouple. Due to the reduced length of the pipe that connects the probe to the control station, to the insulation and to the aspiration-compression speed of the pump, it has been found experimentally that the temperature differences between the mixture within the central reservoir and the probe are lower than 0.1° C.

The advantages of such an apparatus with respect to the preceding thermostimulation devices are evident:

temperature and thermal capacitance of the probe are parameters that can be set forth by the operator: indeeed, the mixture flow is interrupted after a time sufficient for the total filling of the probe when the probe reaches the desired temperature; thus at the time t₀, in the case of cooling, there will be a temperature of 5° C. of the probe, the desired temperature of cryostimulation, or there will be a temperature of +40° C. for investigations based on thermostimulation by means of hot stimulus. Moreover, as from the time t₀ to the time t₁ there is no continuous recycling of liquid inside the probe, it is possible to have a controlled and repeatable thermal exchange between the surface of the probe and the skin of the patient, employing said small rubber balloons of different volumes as the case may be, preferably between 100 and 500 ml. In all cases, the probe is perfectly manoeuverable as it is light and is connected to the supplying station through flexible pipes;

the thermal capacitance of the probe, even in the presence of a 100 ml rubber balloon is anyway capable of hypothermalizing or of hyperthermalizing the cutaneous and the subcutaneous tissue by some cubic centimetres;

the solution is thermostated at the temperature from 0° C. to +50° C., however the maximum cooling that is caused to occur at the head of the probe during cryostimulation is usually of +5° C., whereas for heating (in the hot stimulation) a temperature of 40° C. is employed. Such thermostimulation values have been set forth experimentally, after showing that such values do not cause thermal reflections at a distance or local reactive hyperaemia phenomena.

The disclosure of the present finding will be more easily understood and followed by reference to the enclosed drawings that represent just as a non-limitative example a preferred embodiment of the invention. In the drawings:

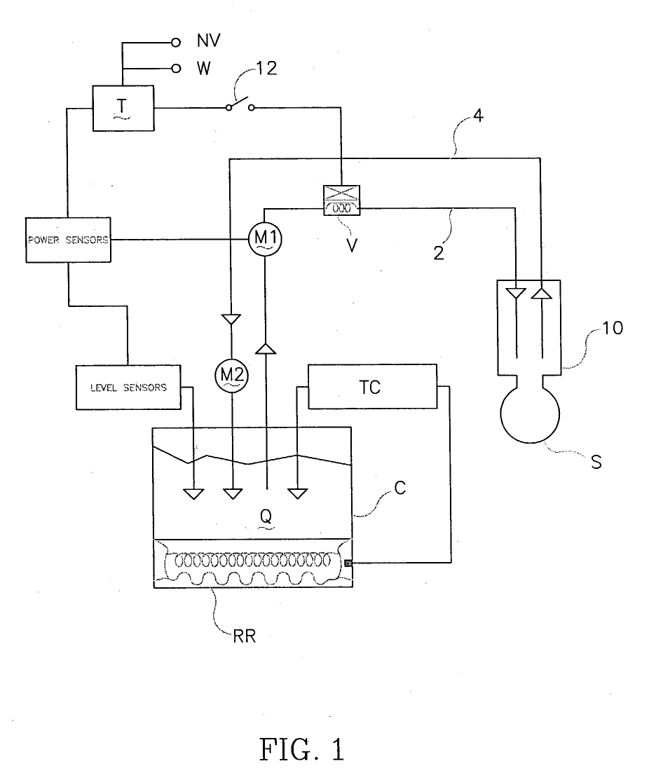

FIG. 1 is a general layout of the thermostimulation apparatus according to the present finding;

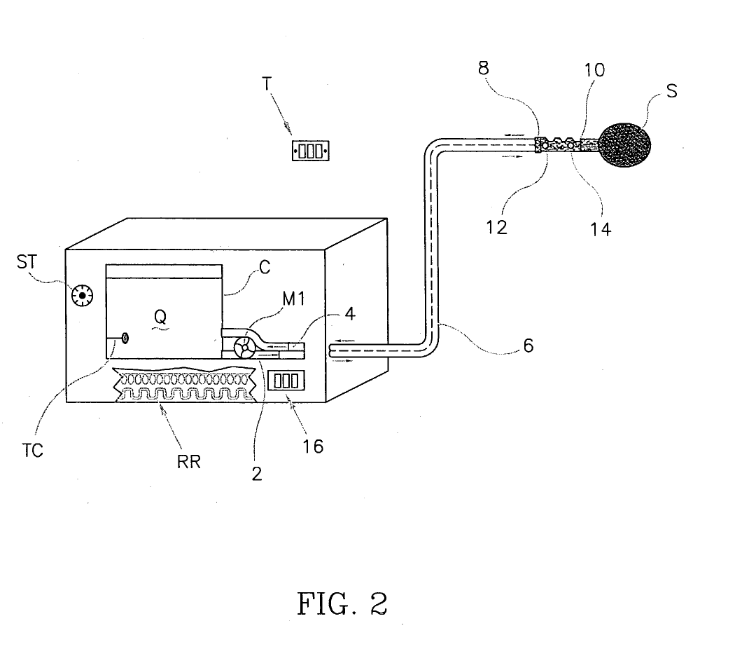

FIG. 2 shows schematically the thermostimulation apparatus as a whole.

With reference to FIG. 1, the apparatus comprises a central unit that comprises a reservoir or tank C containing an amount Q of a 50% solution of distilled water-ethyl alcohol that, by means of an electronic central station which is connected to a double electric circuit and to freon gas RR, is thermostated at a temperature from 0° C. to +50° C., this temperature being set forth previously by means of a variable-step switch ST.

A pump system M1 causes next, through the valve V, the liquid to flow into a first conduit 2 of an insulated flexible pipe 6, at whose end a probe S is inserted, the probe being in the shape of a small rubber balloon of variable volume, from 100 to 500 ml, which once is filled is contacted with the cutaneous area to be examined. When the test is over, at the time t₁, a second pump M2 causes the liquid to flow back, at a command signal from the operator, from the probe S to the reservoir C, through a second conduit 4.

The pipe 6 is connected, preferably by means of a screw-type mechanisms 8, to the small balloon S through a sleeve 10 with a manual handle bearing the starting and valve V control button 12 of the pump M1. A second control switch 14 drives the motor M2 for emptying the probe.

The temperature of the liquid contained within the reservoir C and the application time of the thermal stimulus are pointed out on suitable monitors 16. The temperature of the liquid within the small balloon S is controlled thermographically before the examination, by detecting temperature differences with respect to the central reservoir C that in experimental studies are estimated to be of values lower than 0.1° C.

The opening time of the valve V, such valve being controlled through the button 12 by the operator, is adjusted by means of the timer T. Two pilot lamps show whether the system is on or off.

As already mentioned previously, a thermocouple TC (copper-constantan) is dipped into the reservoir C of the mixture, the thermocouple recording the temperature of the mixture itself and controlling the heating/cooling systems RR for re-establishing the temperature set forth previously. By means of a suitable insulation of the conduits 2 and 4 within the flexible pipe 6, and keeping into account its reduced length (about 1.5 m) it was possible to find experimentally that the temperature of the liquid within the small balloon S, which is checked thermographically before the examination, shows differences with respect to the temperature of the central reservoir C which are lower than 0.1° C.

The operation of the apparatus is very simple: the patient is placed at a fixed distance of 70 cm from the thermograph, in a position which is suitable for the examination of the place of his/her lesions (i.e., standing or seated on a bed). Starting the examination through a thermostimulation technique, the small balloon of the probe is manually put in contact with the area where there is the lesion, with a temperature of +5° C. for a time of 20″ in the case of cryostimulation, and of +40° C. in the case of hot stimulation.

At that point, the values of the thermal recovery times (TRT) of the lesion or of the cutaneous area under examination are recorded. The evaluation of the times in minutes/seconds occurs in real time on the observer's side who checks on the monitor of the thermograph the recovery of the basic isothermals after interruption of the thermal stimulus (time t₁).

A preferred embodiment of this finding has been disclosed herein above: accordingly, it is to be understood that numerous variants can be introduced by those who are skilled in the art which will be included within the realm of the object and scope of this invention, when they are aimed at obtaining the same objectives.

1. A thermostimulating device that can be employed for the thermographic investigation in dermatology, oncology, angiology and capillaroscopy, said device being characterized in that it comprises:
a thermogenic probe that is to contact the cutaneous area where the lesion to be examined is present, said probe comprising a manual handle and a small balloon featuring a surface of controlled thermal exchange, which surface is taken to the stimulation temperature before the contact with the skin by means of a liquid mixture flowing from a reservoir in which said heating/cooling mixture is kept at a stabilized temperature; said reservoir being connected to said probe through separated and insulated in-flow and back-flow pipes; p1 a valve for intercepting said mixture within the in-flow pipe;
a timer for timing the opening of said valve in order to determine the passage of a specific volume of the refrigerating/heating mixture;
a first pump for drawing said mixture out of said reservoir and sending it into the probe;
a second pump for drawing said mixture out of said probe and for sending it again back to the reservoir;
first control members placed on the manual handle of the probe, which can be manoeuvered by the operator, for opening the valve for introduction of the mixture into said probe, and for starting the pump for said introduction in order to take the probe itself to a pre-established temperature;
second control members for emptying the probe.

2. A thermostimulating device that can be employed for thermographic investigation in dermatology, oncology, angiology and capillaroscopy, as claimed in the preceding claim, characterized in that said refrigerating liquid mixture is a 50% mixture of water and alcohol.

3. A thermostimulating device according to claim 1, characterized in that for keeping a pre-established temperature of the probe a thermocouple is provided that records the temperature of the liquid mixture inside the reservoirand controls the heating/cooling system that is present within the reservoir itself, in order to take the mixture again back to a temperature (T+t) that corresponds to the pre-established temperature (T) of the probe, increased by the average temperature variation (t) of the liquid mixture on its way from the reservoir to the probe.

4. A thermostimulating device according to the preceding claims, characterized in that the probe is made up of a sleeve bearing a manual handle on which a small flexible, thin rubber balloon, of different volumes as the case may be, can be screwed.

5. A thermostimulating device according to the preceding claims, characterized in that the timer that controls the inlet flow time of the mixture into the probe can be adjusted manually, according to the size of the probe itself.

6. A thermostimulating device according to the preceding claims, characterized in that the volume of the small rubber balloon that makes up the probe is comprised between 100 and 500 ml.
